(19) United States
(12) Patent Application Publication
(10) Pub. No.: US 2017/0119329 A1
(43) Pub. Date: May 4, 2017

(54) REAL-TIME PATIENT IMAGE OVERLAY DISPLAY AND DEVICE NAVIGATION SYSTEM AND METHOD

(71) Applicant: General Electric Company, Schenectady, NY (US)

(72) Inventors: Adrian F. Warner, Wauwatosa, WI (US); James V. Miller, Niskayuna, NY (US); Claudio P. Mejia, Wauwatosa, WI (US); Daniel R. Schneidewend, Wauwatosa, WI (US); Hao Lai, Niskayuna, NY (US); Nicholas P. Nekich, Wauwatosa, WI (US)

(21) Appl. No.: 14/924,863

(22) Filed: Oct. 28, 2015

(51) Int. Cl. A61B 6/00; A61B 6/04

(52) U.S. Cl. CPC A61B 6/462; A61B 6/0407

(57) ABSTRACT

An interventional radiology system includes an imaging system for generating an image of anatomy of a patient and a catheter within the anatomy and a display system operable to display the image as a projection onto the patient, proximate and aligned with the patient anatomy. An interventional radiology method includes generating an image of anatomy of a patient and a catheter within the anatomy using an imaging system, and displaying the image as a projection onto the patient, proximate and aligned with the patient anatomy.

### FIELD

The disclosed exemplary embodiments relate generally to interventional radiological procedures, and more particularly to displaying images during these procedures.

### BACKGROUND

Interventional radiology generally uses medical imaging techniques such as computer tomography, magnetic resonance, X-ray and fluoroscopy for diagnostic and therapeutic functions.

As part of some interventional radiology procedures, a catheter may be guided to a site within the body for making measurements, retrieving samples, or for performing therapeutic actions, for example, angioplasty, ablation, embolization or other therapeutic actions. The catheter is generally a thin tube which is guided through the anatomy of the patient, for example, through the arterial or venous systems.

U.S. Pat. No. 7,050,845 discloses a system for projecting radioscopic images over video images of a patient. A camera is attached to the rear of a display with a tracking device. When the display is held over a patient, the camera provides a video displayed image of the exterior of an area of the patient while a radioscopic image of the same area is projected onto the video display. The radioscopic image is registered with the video displayed image to create a 1:1 representation of the two images in overlap.

U.S. Pat. No. 7,978,825 discloses an anatomical marker for more effectively orienting a fluoroscopy image to a patient's body position. The marker includes left and right side indicators as well as antero-posterior and postero-posterior indicators.

During catheterization procedures, a physician generally stands up straight and displayed images of the patient's anatomy and other information are provided directly ahead, usually at eye level. However, in this position, the physician must translate his or her movements as seen on the display ahead to actual movements in a different plane. In order to see the physician's hands, the catheter navigation, the scanned view of the anatomy, and the patient, the physician must repeatedly change positions between looking down at the patient and looking forward at the display.

It would be advantageous to provide physicians with the ability to view their hands, the catheter location, patient anatomy, and the patient in the same field of view, and in the same ocular plane.

### SUMMARY

In at least one embodiment, an interventional radiology system includes an imaging system for generating an image of anatomy of a patient and a catheter within the anatomy, and a display system operable to display the image as a projection onto the patient, proximate and aligned with the patient anatomy.

The display system may be operable to display the image offset from the patient anatomy.

The display system may be operable to display the image over the patient anatomy.

The interventional radiology system may include one or more projectors for projecting the image onto the patient.

The interventional radiology system may include one or more projectors for projecting the image proximate the patient anatomy.

The interventional radiology system may include a substrate positioned closely above the patient anatomy onto which the image is projected.

The interventional radiology system may include at least one position indicator projector for projecting a catheter position indicator in the image.

The display system may comprise a monitor.

The monitor of the display system may include a flexible display.

The display system may include a head mounted display assembly.

In another disclosed embodiment, an interventional radiology method includes generating an image of anatomy of a patient and a catheter within the anatomy using an imaging system, and displaying the image as a projection onto the patient, proximate and aligned with the patient anatomy.

The interventional radiology method may include displaying the image offset from the patient anatomy.

The interventional radiology method may include displaying the image over the patient anatomy.

The interventional radiology method may include projecting the image onto the patient.

The interventional radiology method may include projecting the image proximate the patient anatomy.

The interventional radiology method may include projecting the image onto a substrate positioned closely above the patient anatomy.

The interventional radiology method may include projecting a catheter position indicator in the image.

The interventional radiology method may include displaying the image on a monitor positioned closely above the patient anatomy.

The interventional radiology method wherein the monitor comprises a flexible display.

The interventional radiology method may include displaying the image using a head mounted display assembly.

Some of the disclosed embodiments are directed to an interventional radiology system including an imaging system for generating an image of anatomy of a patient and a catheter within the anatomy, a first display system, remote from the patient anatomy being imaged, operable to display the image and additional information, and a second display system operable to display the image as a projection onto the patient, proximate and aligned with the patient anatomy.

The second display system may be operable to display the image on the patient and offset from the patient anatomy.

The second display system may be operable to display the image on the patient and over the patient anatomy.

The interventional radiology system may include one or more projectors for projecting the image proximate and aligned with the patient anatomy.

The interventional radiology system may include a substrate positioned closely above the patient onto which the image is projected.

The second display system may comprise a monitor.

The monitor of the second display system may include a flexible display.

The second display system of the interventional radiology system may include a head mounted display assembly.

### BRIEF DESCRIPTION OF THE DRAWINGS:

The foregoing and other aspects of the disclosed embodiments are made more evident in the following Detailed Description, when read in conjunction with the attached Drawing Figures, wherein:

FIG. 1 shows a diagram of a catheterization system according to the disclosed embodiments;

FIG. 2 shows a block diagram of an exemplary control system;

FIGS. 3A-3D show different projector mountings according to the disclosed embodiments;

FIG. 4 shows a portion of an exemplary catheter according to the disclosed embodiments;

FIG. 5 shows an example of an image illustrating a catheter within a patient according to the disclosed embodiments;

FIGS. 6 and 7 show diagrams of catheterization systems with alternate display systems; and

FIGS. 8A and 8B show diagrams of a head mounted display system.

### DETAILED DESCRIPTION

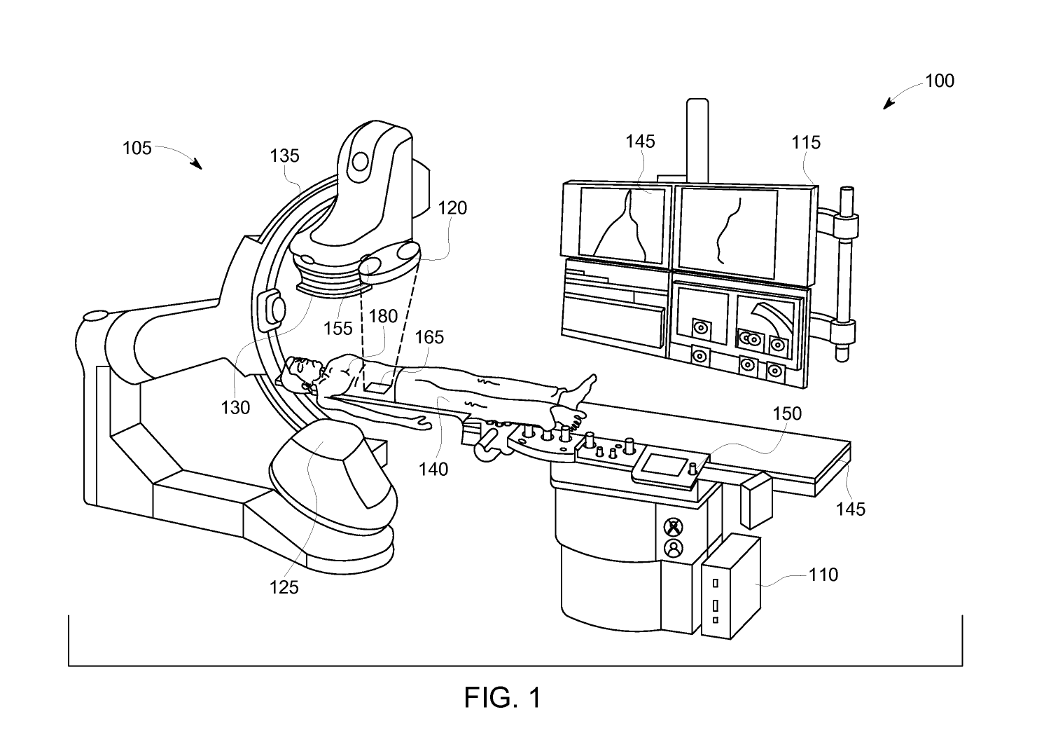

FIG. 1 shows a diagram of an interventional radiology system 100. The system generally includes an imaging system 105, a control system 110, a first display system 115, and a second display system 120 for projecting an image 165 onto a patient 140. The imaging system may include a source 125 of radiation and a radiation detector 130. The source 125 and detector 130 may be commonly mounted on a gantry 135. In some embodiments, the source 125 and detector 130 may be mounted independently on separate positioning assemblies that may be commonly or independently controlled. The source 125 may project radiation, for example, x-rays, through a patient 140 toward the detector 130, and the detector 130 may provide image data to the control system 110. The patient 140 may be positioned in a movable bed 145, also referred to as a couch. The gantry 135, source 125, detector 130, and movable bed 145 may be operated by the control system 110. While the disclosed embodiments are described in the context of an x-ray imaging system, it should be understood that the imaging system 105 may include one or more of an intracardiac ultrasound system, or a cardiac mapping or electrophysiology recording system in any combination thereof.

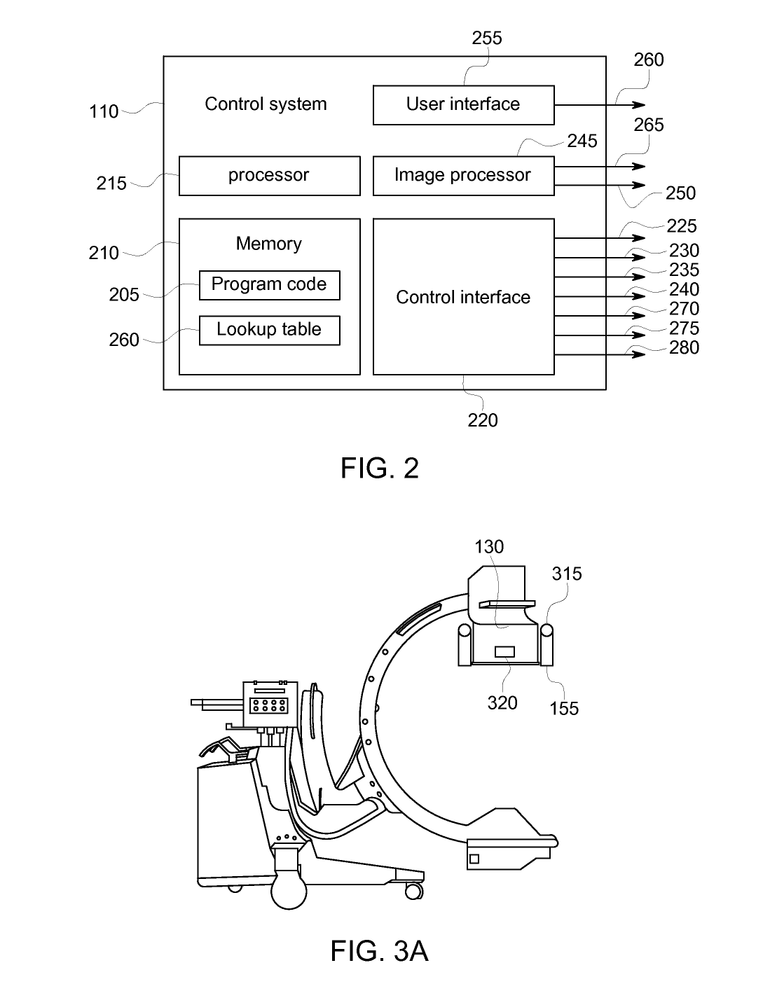

A schematic block diagram of an exemplary control system 110 is shown in FIG. 2. The control system 110 generally includes computer readable program code 205 stored on at least one computer readable medium for carrying out and executing the process steps described herein. The computer readable medium may be a memory 210 of the control system 110. In alternate aspects, the computer readable program code may be stored in a memory external to, or remote from, the control system 110. The memory 210 may include magnetic media, semiconductor media, optical media, or any media which is readable and executable by a computer.

The control system 110 may also include a processor 215 for executing the computer readable program code 205. In at least one aspect, the control system 110 may include one or more input or output devices, including a control interface 220 that provides bidirectional signals for exchanging information with and for controlling one or more devices of the interventional radiology system 100. For example, the control interface 220 may provide control signals for the imaging system 105 including control signals 225 for the source 125, control signals 230 for the detector 130, and control signals 235 for the gantry 135. The control interface may further provide control signals 240 for the movable bed 145 for positioning the patient 140.

The control system 110 may control the position of the gantry 135, the frequency and amount of radiation produced by the source 125, the sensitivity of the detector 130, and the position of the patient 140 in order to facilitate catheterization procedures. Signals from the detector 130 may be sent to the control system 110 for processing. Other control signals may include signals from the patient, for example, respiratory signals 270 or electrocardiograph signals 275.

The control system 110 may include an image processor 245 for processing the signals to produce an output 250 of real time 2D or 3D images of the scanned area of the patient 140. The output 250 of the real time 2D or 3D images may be sent to the first display system 115 for viewing. The first display system 115 is generally located remotely, for example up and away, from the patient anatomy being imaged and may include one or more monitors 145 having a suitable resolution and size for displaying various patient views and patient information for the physician.

The control system 110 may also include a user interface 255 that provides control signals 260 to a user console 150 to allow a physician to provide input for controlling the components of the interventional radiology system 100.

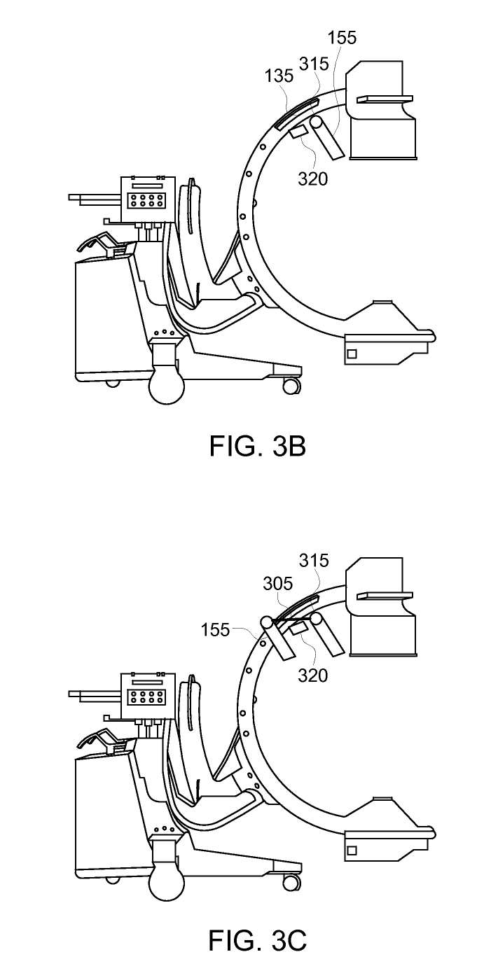

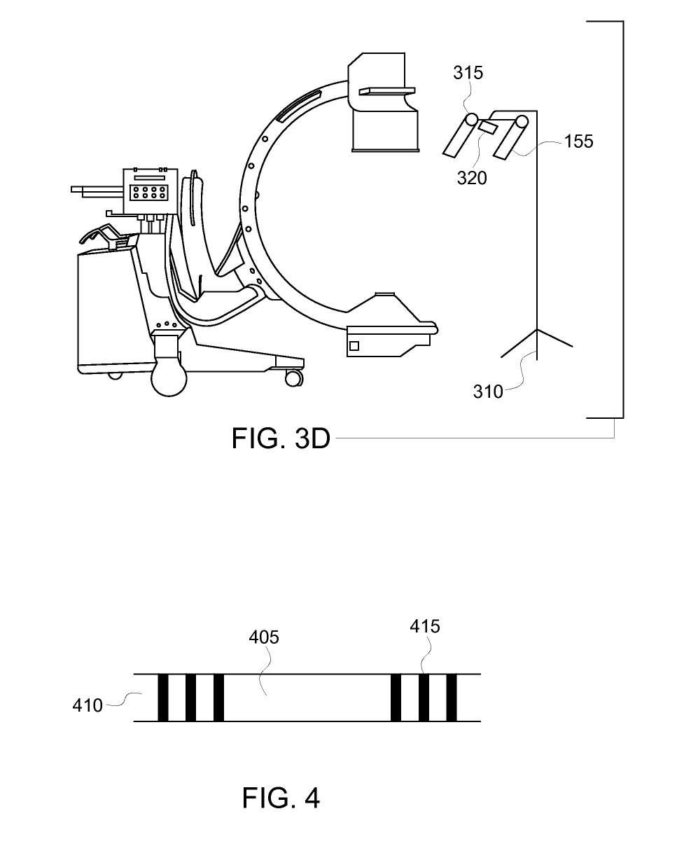

According to the disclosed embodiments, the interventional radiology system 100 further includes a second display system 120 for providing real time images during a catheterization procedure. The images may be color, monochrome, or a combination of color and monochrome. In at least one embodiment, the second display system 120 includes one or more projectors 155. The projectors 155 may be mounted around or on the detector 130 as shown in FIG. 3A and may receive image data from signals 265 of the image processor 245 shown in FIG. 2. As shown in FIG. 3B, the projectors 155 may alternately be mounted on the gantry 135, on a sub gantry 305 as shown in FIG. 3C, or optionally on a separate stand 310, as shown in FIG. 3D. The projectors 155 may be mounted on any suitable structure so long as the projectors 155 may be positioned to project the image 165 onto the patient 140. The image 165 may provide a 2D or 3D view of a catheter and the patient anatomy through which the catheter is being manipulated, directly over or above the patient's torso. As a result, the physician is presented with a view of the catheter and patient anatomy that is aligned with the patient and with the physician's movements. In some embodiments, the projectors 155 may be configured to project an image onto or proximate the patient 140 in the form of a hologram. The projectors 155 may also be mounted on two or three axis gimbal mounts 315 to enable the image 165 from the projectors 155 to be shifted to a desired location on the patient 140. In some embodiments, at least one of the projectors 155 may be used to project a position indicator within the image 165.

FIG. 4 shows a portion of an exemplary catheter 405 according to the disclosed embodiments. The catheter 405 may have one or more lumens 410 and may also include one or more radiopaque or radio opaque markers 415 made of, for example, thin walled metal tubes for visibility during imaging.

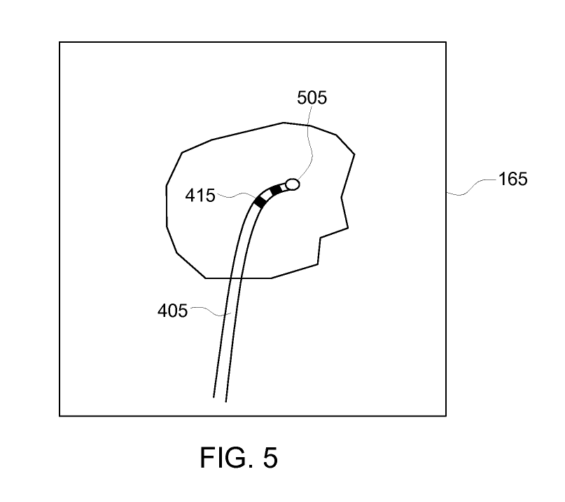

FIG. 5 shows an example of image 165 illustrating the catheter 405 within the patient 140. The second display system 120 may provide the image 165 of the catheter and the anatomy aligned with, or having the same orientation as, the patient anatomy in an offset mode or a superimposed mode. In the offset mode, the image 165 may be projected onto the patient 140 anatomy aligned with, or having the same orientation as the patient anatomy but offset in one or more directions from the actual location of the catheter 405 in the patient 140. This may be advantageous during procedures where the detector 130 may block the physician's view of the catheterization area. In the superimposed mode, the image 165 may be aligned with the patient anatomy and may be projected directly over the patient such that features in the image of the patient anatomy are projected vertically over the actual features in the patient as the catheter 405 progresses through the body of the patient 140. For example, the image 165 may be aligned with one or more of the radio opaque markers 415 in the catheter 405 and may follow the radio opaque markers 415 along the body 140 as the catheter moves.

As mentioned above, at least one of the projectors 155 may be used to project a position indicator within the image 165. FIG. 5 also shows an exemplary position indicator 505. In this example, the position indicator 505 shows the position of a tip of the catheter 405, however, the position indicator may show an outline of the catheter, the position of one or more radio opaque markers, or any other aspect of the catheter. The position indicator 505 may be a light beam projected onto the image 165 such as a laser beam or any suitable light beam and may have any suitable color.

The image 165 may be processed using one or more of the respiration signals 270 and electrocardiograph signals 275 to provide a stable image unaffected by movement of the patient or movement associated with the patient's respiration or cardiac activity.

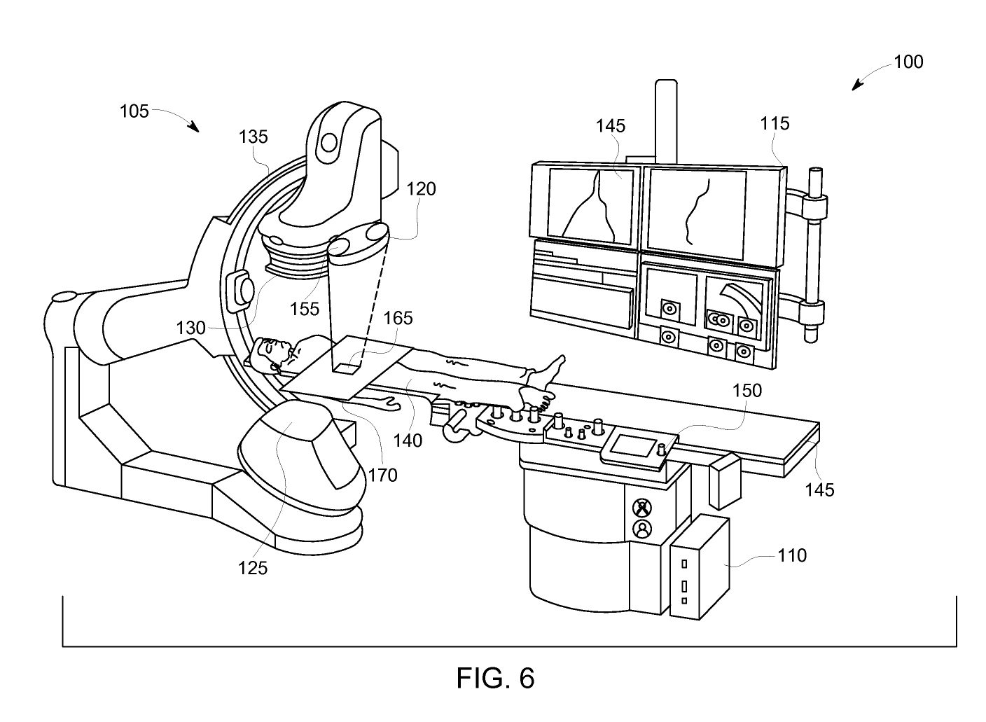

FIG. 6 shows a diagram of the interventional radiology system 100 where the image is projected onto a substrate 170 over the patient 140. The substrate 170 may be located over or under a drape during the catheterization procedure and may provide a screen for the image 165. The substrate 170 may be constructed of one or more of a thin, stiff, lightweight, flat material. In some embodiments, the substrate may be transparent, while in other embodiments the substrate may be opaque, for example, foam board, or any suitable material. The substrate 170 may be mounted without contacting the patient or may be otherwise mounted to be unaffected by patient movement. In embodiments where the imaging system 105 is an x-ray imaging system, the substrate 170 may be radio translucent or transparent and unaffected by radiation from the source 130.

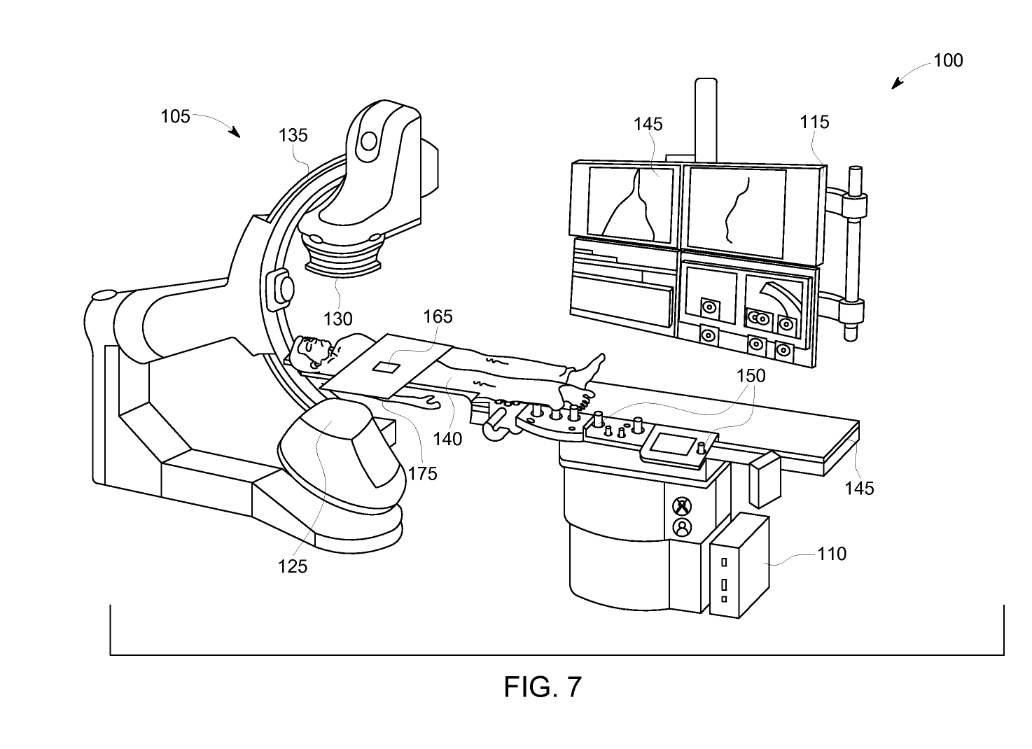

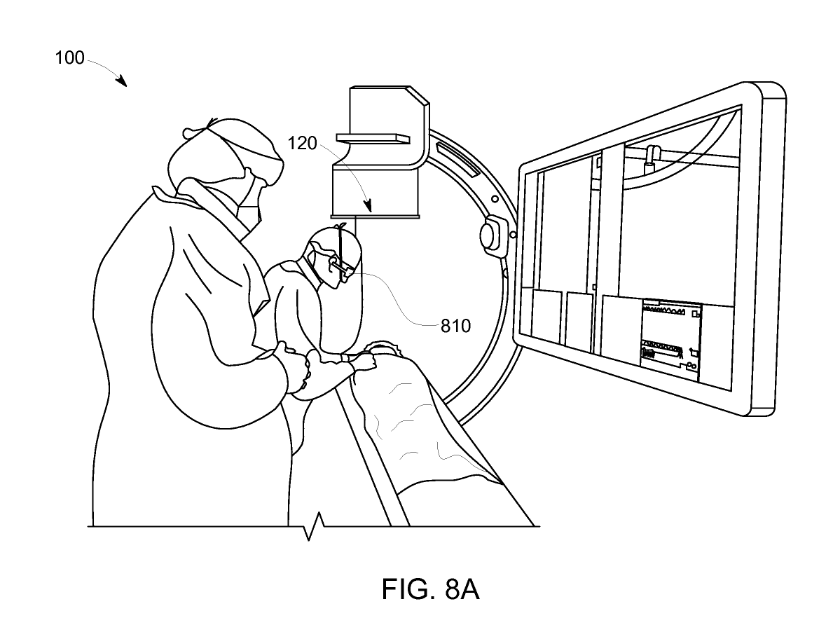

FIG. 7 shows a diagram of the interventional radiology system 100 where, in another embodiment, the second display system 120 includes a monitor 175 mounted over the patient 140. . In some embodiments, the monitor 175 may have a rigid construction, while in other embodiments the monitor 175 may be implemented as a flexible or rollable display that may be lightweight and in some instances, conform to contours of the patient. For example, the monitor 175 may include display circuitry layered onto a bendable substrate, such as organic light emitting diodes or active matrix organic light emitting diodes deposited on a thin film plastic polymer substrate. The monitor 175 may be driven by signals 265 of the image processor 245 and may include a video display utilizing one or more electroluminescent, plasma, liquid crystal, surface conduction, field emission, and nanotube devices, or any other suitable display technology that does not adversely affect, and is not adversely affected by the imaging system 105.

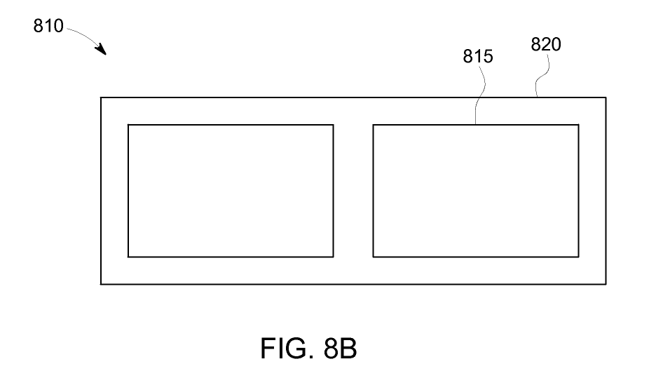

FIG. 8A shows a diagram of the interventional radiology system 100 where, in another embodiment, the second display system 120 includes a head mounted display assembly 810, wearable by a physician. As shown in FIG. 8B, the head mounted display assemblies 810 may include one or more displays 815 mounted on a support structure 820, that when worn on the head, positions the displays 815 in front of the wearers eyes. The displays 815 may be cathode ray tube, liquid crystal display, light emitting diode, organic light emitting diode, or projection displays or any other suitable displays for presenting the information provided by the second display system 120, and may be driven by signals 265 of the image processor 245. The displays 815 may provide images derived from the signals 265 and in some embodiments, may superimpose the images over a real world view, for example, as an augmented reality view. The displays 815 may provide a 2D, 3D, or holographic view of a catheter and the patient anatomy through which the catheter is being manipulated, similar to the other display techniques described above. In some embodiments, the head mounted display assembly 810 may be a virtual reality headset.

Calibration may be required to maintain registration between the second display system 120 and the patient anatomy to compensate for, for example, patient movement, catheter movement, and changes in patient anatomy. The location of the movable bed 145 and the location of the gantry 135 may be monitored by the control system using control signals 240 and 235, respectively and may be used as part of the registration process. Referring to FIGS. 3A-3D, a camera 320 may be mounted on the detector 130, gantry 135, sub gantry 305, stand 310, or any suitable structure that provides the camera 320 with a view of the image 165. The camera 320 may provide a representation of the image 165 to the control system through control signals 280 (FIG. 2). The control system 110 may then correlate the image from the camera 320, image data from the detector 130, the location of the movable bed 145, and the location of the gantry 135 to adjust the location of image 165 with respect to the patient 140. The registration may be further adjusted using one or more of the respiration signals 270 and electrocardiograph signals 275. As shown in FIG. 1, an anatomical radio opaque marker 180 that is visible to both the imaging system 105 and the camera 320 may be applied to the patient 140 in order to enhance the registration.

It should be noted that the second display system 120 may generally provide a supplemental, backup display in the event the first display system 115 fails.

The disclosed embodiments advantageously provide a human factors improvement for the physician. The disclosed second display system gives the physician the ability to view the catheter location, the patient anatomy and patient in the same field of view. The physician is also able to view and feel the catheter in the same ocular plane as the patient anatomy, while previously the physician was required to look at displays positioned up and substantially straight ahead. The disclosed embodiments may reduce fatigue on the physician and provide a view the physician's hands, the catheter navigation and the anatomy in one field of view.

Various modifications and adaptations may become apparent to those skilled in the relevant arts in view of the foregoing description, when read in conjunction with the accompanying drawings. However, all such and similar modifications of the teachings of the disclosed embodiments will still fall within the scope of the disclosed embodiments.

Furthermore, some of the features of the exemplary embodiments could be used to advantage without the corresponding use of other features. As such, the foregoing description should be considered as merely illustrative of the principles of the disclosed embodiments and not in limitation thereof.

1. An interventional radiology system comprising:
an imaging system for generating an image of anatomy of a patient and a catheter within the anatomy; and
a display system operable to display the image as a projection onto the patient, proximate and aligned with the patient anatomy.

2. The interventional radiology system of claim 1, wherein the display system is operable to display the image offset from the patient anatomy.

3. The interventional radiology system of claim 1, wherein the display system is operable to display the image over the patient anatomy.

4. The interventional radiology system of claim 1, comprising one or more projectors for projecting the image onto the patient.

5. The interventional radiology system of claim 1, comprising one or more projectors for projecting the image proximate the patient anatomy.

6. The interventional radiology system of claim 5, comprising a substrate positioned closely above the patient anatomy onto which the image is projected.

7. The interventional radiology system of claim 1, comprising at least one position indicator projector for projecting a catheter position indicator in the image.

8. The interventional radiology system of claim 1, wherein the display system comprises a monitor.

9. The interventional radiology system of claim 8, wherein the monitor comprises a flexible display.

10. The interventional radiology system of claim 1, wherein the display system comprises a head mounted display assembly.

11. An interventional radiology method comprising:
generating an image of anatomy of a patient and a catheter within the anatomy using an imaging system; and
displaying the image as a projection onto the patient, proximate and aligned with the patient anatomy.

12. The interventional radiology method of claim 11, comprising displaying the image offset from the patient anatomy.

13. The interventional radiology method of claim 11, comprising displaying the image over the patient anatomy.

14. The interventional radiology method of claim 11, comprising projecting the image onto the patient.

15. The interventional radiology method of claim 11, comprising projecting the image proximate the patient anatomy.

16. The interventional radiology method of claim 11, comprising projecting the image onto a substrate positioned closely above the patient anatomy.

17. The interventional radiology method of claim 11, comprising projecting a catheter position indicator in the image.

18. The interventional radiology method of claim 11, comprising displaying the image on a monitor positioned closely above the patient anatomy.

19. The interventional radiology method of claim 18, wherein the monitor comprises a flexible display.

20. The interventional radiology method of claim 11, comprising displaying the image using a head mounted display assembly.

21. An interventional radiology system comprising:
an imaging system for generating an image of anatomy of a patient and a catheter within the anatomy;
a first display system, remote from the patient anatomy being imaged, operable to display the image and additional information; and
a second display system operable to display the image as a projection onto the patient, proximate and aligned with the patient anatomy.

22. The interventional radiology system of claim 21, wherein the second display system is operable to display the image on the patient and offset from the patient anatomy.

23. The interventional radiology system of claim 21, wherein the second display system is operable to display the image on the patient and over the patient anatomy.

24. The interventional radiology system of claim 21, wherein the second display system includes a substrate positioned closely above the patient for displaying the image.

25. The interventional radiology system of claim 21, wherein the second display system comprises a monitor.

26. The interventional radiology system of claim 25, wherein the monitor comprises a flexible display.

27. The interventional radiology system of claim 21, wherein the second display system comprises a head mounted display assembly.
